(19) United States
(12) Patent Application Publication
(10) Pub. No.: US 2020/0299214 A1
(43) Pub. Date: Sep. 24, 2020

(54) PRODUCTION OF ETHANOL FROM CARBON DIOXIDE AND HYDROGEN

(71) Applicant: SABIC Global Technologies B.V., Bergen op Zoom (NL)

(72) Inventors: Balamurugan VIDJAYACOUMAR, Thuwal (SA); Vinu VISWANATH, Thuwal (SA); Mohammed BABKOOR, Thuwal (SA); Bedour AL SABBAN, Thuwal (SA); Khalid ALAHMADI, Thuwal (SA); Khalid ALMUSAITEER, Thuwal (SA); Khalid ALBAHILY, Thuwal (SA)

(21) Appl. No.: 16/824,264

(22) Filed: Mar. 19, 2020

Related U.S. Application Data
(60) Provisional application No. 62/821,525, filed on Mar. 21, 2019.

(51) Int. Cl. C07C 29/34; B01J 23/755; B01J 37/18; B01J 21/08; B01J 37/04; B01J 37/03; B01J 37/02; B01J 37/08

(52) U.S. Cl. CPC C07C 29/34; B01J 23/755; B01J 37/18; B01J 37/08; B01J 37/04; B01J 37/035; B01J 37/0236; B01J 21/08

(57) ABSTRACT

Processes for producing alcohols are described. The processes involve contacting a reactant feed that includes a first alcohol with a metal oxalate salt and a Cu-based catalyst under conditions sufficient to produce a product composition that includes a second alcohol. The second alcohol has a higher carbon number than the first alcohol.

### CROSS REFERENCE TO RELATED APPLICATIONS

This application claims the benefit under 35 U.S.C. § 119 of U.S. provisional application Ser. No. 62/821,525 filed Mar. 21, 2019, incorporated herein by reference in its entirety.

### BACKGROUND OF THE INVENTION

### A. Field of the Invention

The invention is directed to the production of alcohols. In particular, the invention concerns a process for producing ethanol from methanol using a metal oxalate salt and a copper-based catalyst under a CO₂ and hydrogen atmosphere.

### B. Description of Related Art

Ethanol is an important commodity chemical that is used in various industrial processes. By way of example, because ethanol can readily dissolve in water and other organic compounds, it is used as an ingredient in a range of products from personal care and beauty products to paints and varnishes to fuel. With respect to fuel, ethanol can be blended with gasoline which can be used in motor vehicles. Many gasoline stations provide a blended fuel, which typically is 10 percent ethanol and 90 percent gasoline.

Presently, ethanol is commercially produced naturally as a byproduct of plant fermentation and also can be produced through the hydration of ethylene industrially primarily by the acid-catalyzed (e.g., supported phosphoric acid) hydration of ethylene. Other catalytic methods includes the hydrogenation of CO₂. By way of example, Takagawa (Studies in Surface Science and Catalysis, 1998, 114, 525-528) describes the use of potassium copper zinc iron (K/Cu-Zn-Fe) oxides for hydrogenation of CO₂ to produce ethanol with a 20% selectivity. This method suffers in that temperatures greater than 250° C. are required for the 20% selectivity, and at those temperatures, the catalyst activity diminishes.

### SUMMARY OF THE INVENTION

A discovery has been made that provides an alternate feedstock that can be used for the production of alcohols (e.g., ethanol). The discovery is premised on the decomposition of an oxalate metal salt in combination with a copper (Cu)-based catalyst in the presence of an alcohol to produce a mono alcohol rather than a diol. By way of example, ethanol can be prepared from cesium oxalate. Advantages of the processes of the present invention include the efficient production of alcohols (e.g. ethanol) without out relying on relatively slow and costly plant fermentation processes or the use of valuable chemicals (e.g., ethylene) as feed stock. Further, the processes of the present invention can be performed at temperatures (e.g., less than 250° C., preferably 210° C. to 240° C.) that are less damaging to catalysts when compared with current processes that utilize hydrogenation of CO₂ to produce alcohols.

In one aspect of the present invention, processes for producing an alcohol are described. A process can include contacting a metal oxalate salt with a first alcohol (e.g., methanol) in the presence of a copper (Cu)-based catalyst under conditions sufficient to produce a composition that includes a second alcohol (e.g., ethanol) having an increased carbon number relative to the first alcohol. Contacting conditions can include a carbon dioxide (CO₂) and hydrogen (H₂) atmosphere, a temperature of 200° C. to 250° C., 210° C. to 240° C., or preferably about 220° C., or any combination thereof. The metal oxalate salt to first alcohol molar ratio can be 1 to 5, preferably 3. A molar ratio of the first alcohol (e.g., methanol) to the Cu in the Cu-based catalyst can be 200:1, preferably 150:1, more preferably 100:1. In certain instances, the molar ration of the first alcohol to the Cu in the Cu-based catalyst can be 200:1 to 50:1, preferably 150:1 to 50:1, or more preferable 125:1 to 75:1. The Cu-based catalyst can include nickel (Ni). In some embodiments, the Cu-based catalyst is supported. In a preferred embodiment, the catalyst is a Cu-Ni alloy supported on silica. Prior to contacting the Cu-based catalyst with the first alcohol, a Cu-based catalyst precursor can be contacted with gaseous hydrogen (H₂) under conditions sufficient (e.g., a temperature of 250° C. to 280° C., 260° C. to 270° C., or preferably about 265° C.) to reduce the Cu-based catalyst precursor and form the Cu-based catalyst. The metal oxalate salt can include cesium (Cs), preferably cesium oxalate (Cs₂C₂O₄). In addition to the second alcohol (e.g., ethanol), the composition can further include a diol (e.g., ethylene glycol). A mole ratio of second alcohol to diol can be 10:1 to 20:1. The selectivity to the second alcohol can be at least 10%.

The cesium salt can be cesium oxalate and/or a cesium oxalate/inert material composition. The cesium oxalate can be obtained by contacting a mixture of CO₂ and carbon monoxide (CO) under reaction conditions sufficient to form a composition containing the cesium oxalate. In some instances, the cesium oxalate can be obtained by contacting a mixture of CO₂ and hydrogen (H₂), or a mixture of O₂ and CO with cesium carbonate (Cs₂CO₃), under reaction conditions sufficient to form a composition containing the cesium oxalate. The inert material can be added to the cesium carbonate. Particularly, the reaction conditions for obtaining the cesium oxalate can include a temperature of 200° C. to 400° C., 250° C. to 350° C., preferably 290° C. to 335° C., or most preferably 300° C. to 325° C. In some instances, the reaction conditions for obtaining the cesium oxalate can include providing carbon dioxide at a pressure of 2.0 MPa to 3.0 MPa, preferably about 2.5 MPa, and providing carbon monoxide at a pressure of 1.0 MPa to 3 MPa, preferably about 2.0 MPa. In other instances, the reaction conditions for obtaining the cesium oxalate can include providing carbon dioxide at a pressure of 2.0 MPa to 4.0 MPa, preferably about 3.5 MPa, and providing H₂ at a pressure of 0.05 MPa to 0.5 MPa, preferably about 0.1 MPa. In yet another instance, the reaction conditions for obtaining the cesium oxalate can include providing carbon monoxide at a pressure of 2.0 MPa to 4.0 MPa, preferably about 3.5 MPa, and providing O₂ at a pressure of 0.05 MPa to 4 MPa, 0.1 to 1.5 MPa, or about 0.1 MPa. The process can further include contacting the cesium carbonate with the carbon dioxide at a reaction temperature of 200° C. to 400° C., 250° C. to 350° C., preferably 290° C. to 335° C., or most preferably 300° C. to 325° C., for at least 1 hour to form a cesium carbonate/carbon dioxide reaction mixture and then contacting the cesium carbonate/carbon dioxide reaction mixture with hydrogen. Such a controlled addition of the carbon dioxide and hydrogen can inhibit the formation of sodium formate. In a particular instance, the process can further include isolating the cesium oxalate salt from the product stream prior to converting it to the disubstituted oxalate. Alternatively, the process can be a one-pot synthesis such that it is performed in a single reactor such that cesium oxalate is generated in situ and then contacted with the one or more alcohols and additional CO₂ to produce the desired alcohol.

The following includes definitions of various terms and phrases used throughout this specification.

The term “alkyl group” can be a straight or branched chain alkyl having 1 to 20 carbon atoms. Examples include methyl, ethyl, propyl, isopropyl, butyl, isobutyl, secondary butyl, tertiary butyl, pentyl, isopentyl, neopentyl, hexyl, benzyl, heptyl, octyl, 2-ethylhexyl, 1,1,3,3-tetramethylbutyl, nonyl, decyl, dodecyl, tetradecyl, hexadecyl, octadecyl, and/or eicosyl.

The term “substituted alkyl group” can include any of the aforementioned alkyl groups that are additionally substituted with one or more heteroatom, such as a halogen (F, Cl, Br, I), boron, oxygen, nitrogen, sulfur, silicon, etc. Without limitation, a substituted alkyl group can include alkoxy or alkylamine groups where the alkyl group attached to the heteroatom can also be a substituted alkyl group.

The term “aromatic group” can be any aromatic hydrocarbon group having 5 to 20 carbon atoms of the monocyclic, polycyclic or condensed polycyclic type. Examples include phenyl, biphenyl, naphthyl, and the like. Without limitation, an aromatic group also includes heteroaromatic groups, for example, pyridyl, indolyl, indazolyl, quinolinyl, isoquinolinyl, and the like.

The term “substituted aromatic group” can include any of the aforementioned aromatic groups that are additionally substituted with one or more atom, such as a halogen (F, Cl, Br, I), carbon, boron, oxygen, nitrogen, sulfur, silicon, etc. Without limitation, a substituted aromatic group can be substituted with alkyl or substituted alkyl groups including alkoxy or alkylamine groups.

The term “inert” is defined as a material or chemical that undergo a chemical reaction with the starting materials or product during the course of the reaction.

The terms “about” or “approximately” are defined as being close to as understood by one of ordinary skill in the art. In one non-limiting embodiment, the terms are defined to be within 10%, preferably, within 5%, more preferably, within 1%, and most preferably, within 0.5%.

The terms “wt. %,” “vol. %,” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification. With respect to the transitional phase “consisting essentially of,” in one non-limiting aspect, a basic and novel characteristic of the process of the present invention is the ability to produce an alcohol (e.g., ethanol) by contacting a metal oxalate salt, a lower number alcohol (e.g., methanol) with a Cu-based catalyst.

In the contest of the present invention, at least twenty embodiments are now described. Embodiment 1 is a process for producing an alcohol. The process includes the steps of contacting a metal oxalate salt with a first alcohol in the presence of a copper (Cu)-based catalyst under conditions sufficient to produce a composition containing a second alcohol having an increased carbon number relative to the first alcohol. Embodiment 2 is the process of embodiment 1, wherein the first alcohol is methanol and the second alcohol is ethanol. Embodiment 3 is the process of any one of embodiments 1 or 2, wherein contacting is performed in a carbon dioxide (CO₂) and hydrogen (H₂) atmosphere. Embodiment 4 is the process of any one of embodiments 1 to 3, wherein the Cu-based catalyst contains nickel (Ni). Embodiment 5 is the process of embodiment 4, wherein the Cu-based catalyst is a supported Cu-Ni material, preferably Cu-Ni alloy on a silica support. Embodiment 6 is the process of any one of embodiments 1 to 5, wherein the metal oxalate salt to first alcohol molar ratio is 1 to 5, preferably 3. Embodiment 7 is the process of any one of embodiments 1 to 6, wherein the reaction conditions include a temperature of 200° C. to 250° C., 210° C. to 240° C., or preferably about 220° C. Embodiment 8 is the process of any one of embodiments 1 to 7, wherein the metal oxalate salt contains cesium (Cs). Embodiment 9 is the process of embodiment 8, wherein the metal oxalate salt is cesium oxalate (Cs₂C₂O₄). Embodiment 10 is the process of embodiment 9, wherein the cesium oxalate is obtained by contacting CO₂ and H₂ with cesium carbonate (Cs₂CO₃) under reaction conditions sufficient to form the Cs₂C₂O₄. Embodiment 11 is the process of any one of embodiments 9 to 10, wherein the reaction conditions include a temperature of 250° C. to 400° C., preferably 300° C. to 375° C., more preferably 310° C. to 335° C., most preferably 320° C. to 330° C. Embodiment 12 is the process of any one of embodiments 9 to 11, wherein the reaction conditions include a CO₂ pressure of 1 MPa to 5 MPa, 1.5 MPa to 4 MPa, or preferably about 2 MPa, or both at a pressure of 3 MPa to 40 MPa, and a H₂ pressure of 0.05 to 0.5 MPa. Embodiment 13 is the process of any one of embodiments 8 to 12, wherein the mole ratio of CO₂ and H₂ to Cs₂CO₃ is 100:1 to 300:1, preferably 150:1 to 250:1, more preferably 200:1. Embodiment 14 is the process of any one of embodiments 1 to 13, further including the step of contacting a Cu-based catalyst precursor with gaseous hydrogen (H₂) under conditions sufficient to reduce the Cu-based catalyst precursor and form the Cu-based catalyst prior to contacting the Cu-based catalyst with the first alcohol. Embodiment 15 is the process of embodiment 14, wherein the conditions comprise a temperature of 250° C. to 280° C., 260° C. to 270° C., or preferably about 265° C. Embodiment 16 is the process of any one of embodiments 1 to 15, wherein the composition further includes a diol. Embodiment 17 is the process of embodiment 16, wherein the diol is ethylene glycol. Embodiment 18 is the process of any one of embodiments 16 to 17, wherein a mole ratio of second alcohol to diol is 10:1 to 20:1. Embodiment 19 is the process of any one of embodiments 1 to 18, wherein the selectivity to the second alcohol is at least 10%. Embodiment 20 is the process of any one of embodiments 1 to 19, wherein a molar ratio of the first alcohol to the Cu in the Cu-based catalyst is 200:1, preferably 150:1, more preferably 100:1.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 is a schematic of a system to perform the process of the present invention to produce a higher carbon number alcohol (e.g., ethanol) from a lower number alcohol (e.g., methanol) and a metal oxalate salt (e.g., cesium oxalate).

FIG. 2 is a schematic of a process and system to produce metal oxalates.

While the invention is susceptible to various modifications and alternative forms, specific embodiments thereof are shown by way of example in the drawings. The drawings may not be to scale.

### DETAILED DESCRIPTION OF THE INVENTION

A discovery has been made that provides an elegant solution to the problem of production of ethanol from alternative feed stocks. The discovery is premised on contacting a metal oxalate, Cu-based catalyst, and a first alcohol to produce a second alcohol (e.g., ethanol). The reaction mixture, which includes a first alcohol (e.g., methanol) and carbon dioxide (CO₂) can be contacted with a metal oxalate salt (e.g., cesium oxalate) under reaction conditions sufficient to produce an alcohol (e.g., ethanol) containing composition as shown in overall general reaction equation (1).

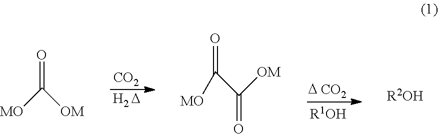

where M is metal cation and R¹ and R² are aliphatic or aromatic groups and R² is at least one carbon number higher than R¹. In some instances, the cesium salt is cesium oxalate, R¹OH is methanol and R²OH is ethanol. These and other non-limiting aspects of the present invention are discussed in further detail in the following sections with reference to the Figures.

### A. System and Processes to Prepare Alcohols

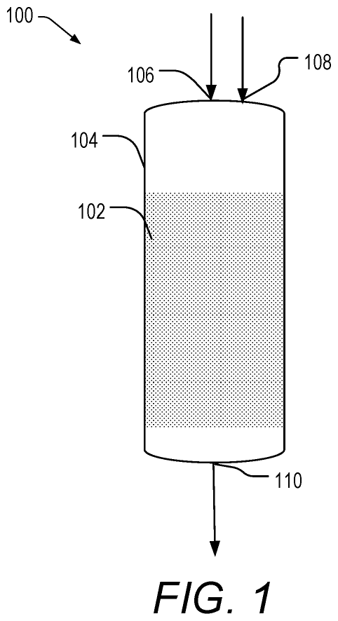

Referring to FIG. 1, a method and system to prepare alcohols (e.g., ethanol) is described. In system 100, a known amount of Cu-based catalyst (e.g., Cu-Ni) 102 can be positioned in reaction unit 104. The Cu-based catalyst can be activated by heating the catalyst under a hydrogen atmosphere. For example, a H₂-containing stream can enter reaction unit 104 via gaseous inlet 106 until the reaction unit is pressurized of 0.2 to 1 MPa, or 0.3 to 0.8 MPa, or at least any one of, equal to any one of, or between any two of 0.2, 0.3, 0.4, 0.5, 0.6, 0.7, 0.8, 0.9 and 1 MPa. Pressurized reaction unit 104 can be heated to a temperature of 250° C. to 300° C., or at least any one of, equal to any one of, or between any two of 250° C., 255° C., 260° C., 265° C., 270° C., 275° C., 280° C., 285° C., 290° C., 295° C., and 300° C. until the catalyst is reduced (e.g., for 1 to 24 hours, 2 to 10 hours, 3 to 8 hours or any range or value there between. After reducing the catalyst, the heating can be stopped and the reaction unit 204 and the catalyst can be cooled to ambient temperature (e.g., 20° C. to 40° C.).

The desired alcohol (e.g., methanol) and metal oxalate (e.g., cesium oxalate) be added to reaction unit 104. The molar ratio of the metal oxalate salt to first alcohol can be 1 to 5, or 1, 2, 3, 4, and 5 or any range or value there between. In a preferred embodiment, the molar ratio is 3. The oxalate salt can be made as described throughout the Specification and/or be purchased from a commercial source. The desired alcohol (e.g., methanol) can be added to reaction unit 104 via liquid inlet 108 to form a composition that includes a metal oxalate salt (e.g., cesium oxalate), an alcohol. CO₂ and H₂ can be added through gaseous inlet 106. The reactor can be pressurized with CO₂ and H₂ to a pressure ranging from 2 MPa to 5 MPa, 3 MPa to 4 MPa, and all ranges and pressures there between (e.g., 2.1 MPa, 2.2 MPa, 2.3 MPa, 2.4 MPa, 2.5 MPa, 2.6 MPa, 2.7 MPa, 2.8 MPa, 2.9 MPa, 3 MPa, 3.1 MPa, 3.2 MPa, 3.3 MPa, 3.4 MPa, 3.5 MPa, 3.6 MPa, 3.7 MPa, 3.8 MPa, 3.9 MPa, 4.0 MPa, 4.1 MPa, 4.2 MPa, 4.3 MPa, 4.4 MPa, 4.5 MPa, 4.6 MPa, 4.7 MPa, 4.8 MPa, or 4.9 MPa). In some embodiments, CO₂ and H₂ are added separately. CO₂ and/or H₂ can be provided to reaction unit 102 at a pressure ranging from 1 MPa to 4 MPa and all ranges and pressures there between (e.g., 1.1 MPa, 1.2 MPa, 1.3 MPa, 1.4 MPa, 1.5 MPa, 1.6 MPa, 1.7 MPa, 1.8 MPa, 1.9 MPa, 2 MPa, 2.1 MPa, 2.2 MPa, 2.3 MPa, 2.4 MPa, 2.5 MPa, 2.6 MPa, 2.7 MPa, 2.8 MPa, 2.9 MPa, 3.0 MPa, 3.1 MPa, 3.2 MPa, 3.3 MPa, 3.4 MPa, 3.5 MPa, 3.6 MPa, 3.7 MPa, 3.8 MPa, 3.9 MPa, or 4 MPa). Preferably, the CO₂ pressure is about 2.5 MPa to 3.5 MPa.

After the addition of the alcohol, H₂, and CO₂ to the reduced catalyst, reaction unit 104 can be heated to a reaction temperature sufficient to promote the metal oxalate salt to react with the alcohol under the CO₂/H₂ atmosphere to produce a second alcohol (e.g., ethanol) containing composition having an increased carbon number from the first alcohol (e.g., (methanol). The reaction temperature can be 115° C. to 200° C., 130° C. to 180° C., or at least, equal to, or between any two of 115° C., 120° C., 125° C., 130° C., 135° C., 140° C., 145° C., 150° C., 155° C., 160° C., 165° C., 170° C., 175° C., 180° C., 185° C., 190° C., 195° C. and 200° C. Preferably, the reaction temperature is about 130° C. Reaction unit 104, and, thus the reaction mixture, can be heated for a time sufficient to react all or substantially all of the metal oxalate salt (e.g., cesium oxalate). By way of example, the reaction time range can be at least 1 hour, 1 hours to 18 hours, 10 hour to 14 hours, 1 to 6 hours or 1 to 2 hours, and all ranges and times there between (e.g., 2 hours, 5 hours, 8 hours, 10 hours, 15 hours, or 17 hours). Preferably, the reaction time is 1 to 18 hours, or 15 hours. The upper limit on temperature, pressure, and/or time can be determined by the reactor used. The reaction temperature can be varied depending on the type of Cu-based catalyst used.

Reaction unit 104 can be cooled and depressurized to a temperature and pressure sufficient (e.g., below 50° C. at 0.101 MPa) to allow removal of the product composition containing the desired alcohol (e.g., ethanol) via product outlet 110. The product composition can be collected for further use. In some instances, the product composition can include diols (e.g., ethylene glycol) in addition to the desired alcohol.

### B. Products

The process of the present invention can produce a product stream that includes a composition containing an alcohol, preferably ethanol, that can be suitable as an intermediate or as feed material in a subsequent synthesis reactions to form a chemical product or a plurality of chemical products (e.g., such as in pharmaceutical products, for the production of oxalic acid and ethylene glycol, or as a solvent or plasticizer). In some instances, the composition includes ethylene glycol. The product composition includes at least 10 wt. %, at least 20 wt. %, at least 30 wt. %, at least 40 wt. %, at least 50 wt. %, at least 60 wt. %, at least 70 wt. %, at least 80 wt. %, at least 90 wt. % or 100 wt. % alcohol with the balance being by-products such as the reactant alcohol (e.g., methanol) and/or a metal bicarbonate (e.g., cesium bicarbonate). A molar ratio of alcohol to diol can be 10:1 to 20:1, or at least any one of, equal to any one of, or between any two of 10:1, 11:1, 12:1, 13:1, 14:1, 15:1, 16:1, 17:1, 18:1, 19:1, and 20:1. The product composition can be purified using known organic purification methods (e.g., extraction, crystallization, distillation, washing, etc.) depending on the phase of the production composition (e.g., liquid or dispersion). In a preferred embodiment, the ethanol can be distilled from the product mixture. Alcohols that can be produced by this method include ethanol, propanol, butanol, pentanol, etc.

### C. Materials

### 1. Cesium Salts and Cesium Oxalate

Cesium salts (e.g., carbonate (Cs₂CO₃)) may be purchased in various grades from commercial sources. Preferably, the cesium salt (Cs₂CO₃) is highly pure and substantially devoid of water. A non-limited commercial source of the cesium salts for use in the present invention includes SigmaMillipore (USA). In some embodiments, Cs₂CO₃ is mixed with an inert material. Non-limiting examples of inert materials include alumina (acidic, basic or neutral), silica, zirconia, ceria, zeolites, lanthanum oxides, or mixtures thereof. In preferred embodiments, the Cs₂CO₃ is mixed with alumina or silica using solid-solid mixing. Providing the Cs₂CO₃ as a Cs₂CO₃/inert material mixture can inhibit the cesium oxalate from forming a melt that requires further processing (e.g., grinding, powdering, etc.) prior to reaction with alcohol to form the disubstituted oxalate of the present invention.

Cesium oxalate can be purchased or prepared as described herein. Cesium oxalate production can be produced in the context of the present invention by contacting a mixture of inert material and a cesium salt (e.g., Cs₂CO₃ and/or CsHCO₃) with an oxygen source and a carbon source under reaction conditions sufficient to form a composition that includes Cs₂C₂O₄. The composition can also include cesium formate (HCO₂Cs) or cesium bicarbonate (CsHCO₃). Formation of a cesium oxalate in the presence of an inert material can inhibit the cesium oxalate from forming a melt that requires further processing (e.g., grinding, powdering, etc.) prior to reaction with other reagents to form various products (e.g., ethanol) especially when the cesium oxalate is generated in situ. The inert material can be any material that does not promote reactions between the gaseous carbon source and the gaseous oxygen source. In some embodiments, the inert material can include at least one metal oxide, charcoal, or a mixture thereof. Non-limiting examples of metal oxides include alumina (acidic, basic, gamma, or neutral), ceria, silica, zirconia, lanthanum oxides, zeolites, or mixtures thereof. In one non-limiting embodiment, alumina and/or silica is used as the inert material. In one particular embodiment, gamma alumina is used as the inert material. In another embodiment, alumina and/or silica is combined with charcoal, and the mixture is used as the inert material. The mass ratio of charcoal to metal oxide can be 0.1:10 to 10:0.1, or 0.2:8, 1:5, 1:1, 2:1, or 3:0.2, preferably 1:1. A mass ratio of inert material to the cesium salt can be 0.1:10 to 10:0.1, or 0.2:8, 0.5:5, 1:1, 2:1, 5:0.2, or 8:0.5. In one non-limiting embodiment, the mass ratio of inert material to the cesium salt can be 1:1, or 0.5:1. In some embodiments, the inert material (e.g., gamma alumina) is added to the cesium carbonate or bicarbonate in the presence of water and mixed under agitation to form a dispersion, slurry, mull, or wet powder of inert material and cesium salt. The water can be removed under vacuum and the resulting powder dried under vacuum at a temperature of 250to 325° C. for 10 minutes to 5 hours, or 15 minutes to 2 hours.

Conventionally, cesium oxalate is generated by the reaction of cesium carbonate with carbon monoxide and carbon dioxide as shown in reaction equation (2).

Cs₂CO₃+CO+CO₂→Cs₂(C₂O₄) (2).

In one alternative process, the cesium oxalate can be generated by the reaction of cesium carbonate with carbon dioxide and H₂ as shown in reaction equation (3) as described in more detail below.

Cs₂CO₃+H₂+CO₂→Cs₂(C₂O₄) (3).

In some embodiments, the carbon dioxide and H₂ are added in a sequential manner as shown in reaction equation (4). The sequential addition of carbon dioxide then hydrogen can inhibit or substantially inhibit the formation of cesium formate (HCO₂Cs). Limiting the formation of cesium formate limits the formation of alkyl formate in subsequent reactions with alcohols. In some instances, cesium formate is not formed in the production of cesium oxalate.

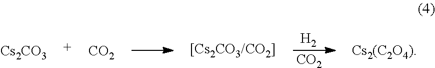

In yet another alternative process, the cesium oxalate can be generated by the reaction of cesium carbonate with carbon monoxide and O₂ as shown in reaction equation (5) as described in more detail below.

Cs₂CO₃+O₂+CO →Cs₂(C₂O₄) (5).

While the above reactions show Cs₂CO₃, the reactions are the same when an inert material is used. With respect to reaction equation (5), and without wishing to be bound by theory, it is believed that the use of molecular oxygen can require lower heat requirements when compared to other processes as the reaction between CO and O₂ is exothermic (free energy change of −61.4 kcal/mol as determined through density functional theory (DFT)). In another embodiment, the oxalate salt (e.g., cesium oxalate) can be produced from oxalic acid and a metal hydroxide (e.g., cesium hydroxide). By way of example, oxalic acid can be mixed with water or another solvent until dissolved. Two molar equivalents of cesium hydroxide can be added to the acidic solution of oxalic acid until full neutralization is achieved (e.g., pH of 6.8 to 7.2). Either the amount of the acid or the base can be in slight excess to ensure completion of neutralization. After the completion of the neutralization, the reaction solution can be concentrated (e.g., vacuum distilled, evaporated) to remove the solvent (e.g., water), and a product can be collected that includes cesium oxalate. In some embodiments, the solution can be concentrated to remove a majority of the solvent (e.g., about 90 to 95 vol. % of the water) and the solution can be cooled to promote crystallization of the cesium oxalate from the solvent. The cesium oxalate can then be isolated (e.g., filtered, centrifuged) and washed thoroughly with ethanol.

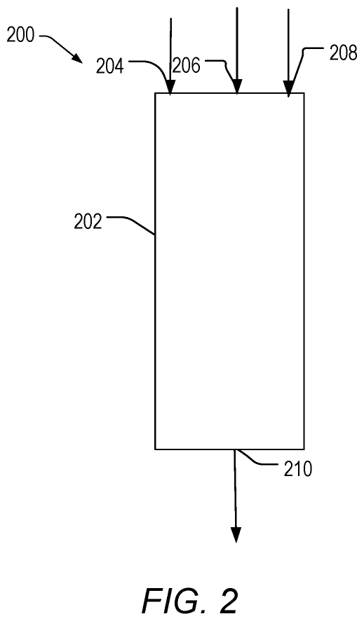

Referring to FIG. 2, a method and system to prepare oxalate salts is described. In system 200, a cesium salt precursor (e.g., cesium carbonate (Cs₂CO₃)) and optional inert material can be provided to a reactor unit 202 via solids inlet 204. CO, CO₂, O₂, or H₂, or any combination thereof can be provided to reactor 202 via gas inlets 206 and 208. By way of example, CO₂ can be provided to reactor 202 via gas inlet 208 and CO or H₂, can be provided to the reactor via gas inlet 206. CO can be provided to reactor 202 via gas inlet 206 and O₂ can be provided to the reactor via gas inlet 208. In embodiments when carbon monoxide is used, the CO can be provided to reactor 202 at a pressure ranging from 1 MPa to 3 MPa and all ranges and pressures there between (e.g., 1.1 MPa, 1.2 MPa, 1.3 MPa, 1.4 MPa, 1.5 MPa, 1.6 MPa, 1.7 MPa, 1.8 MPa, 1.9 MPa, 2 MPa, 2.1 MPa, 2.2 MPa, 2.3 MPa, 2.4 MPa, 2.5 MPa, 2.6 MPa, 2.7 MPa, 2.8 MPa, or 2.9 MPa). Preferably, the CO pressure is about 2 MPa. In other embodiments when H₂ is used, the H₂ can be provided to reactor 202 at a pressure ranging from 0.05 MPa to 0.5 MPa, 0.05 to 0.4 MPa, 0.05 to 0.3 MPa, 0.05 to 0.2 MPa, or 0.05 to 0.1 and all ranges and pressures there between (e.g., 0.05 MPa, 0.06 MPa, 0.07 MPa, 0.08 MPa, 0.09 MPa, 0.1 MPa, 0.11 MPa, 0.12 MPa, 0.13 MPa, 0.14 MPa, 0.15 MPa, 0.16 MPa, 0.17 MPa, 0.18 MPa, 0.19 MPa, 0.20 MPa, 0.21 MPa, 0.22 MPa, 0.23 MPa, 0.24 MPa, 0.25 MPa, 0.26 MPa, 0.27 MPa, 0.28 MPa, 0.29 MPa, 0.30 MPa, 0.31 MPa, 0.32 MPa, 0.33 MPa, 0.34 MPa, 0.35 MPa, 0.36 MPa, 0.37 MPa, 0.38 MPa, 0.39 MPa, 0.40 MPa, 0.41 MPa, 0.42 MPa, 0.43 MPa, 0.44 MPa, 0.45 MPa, 0.46 MPa, 0.47 MPa, 0.48 MPa, 0.49 MPa, or 0.50 MPa). Preferably, the H₂ pressure is about 0.1 MPa. In other embodiments when O₂ is used, the O₂ can be provided to reactor 202 at a pressure ranging from 0.05 MPa to 4 MPa, 0.1 to 1.5 MPa, or about 0.1 MPa. CO₂ can be provided to reactor 202 at a pressure ranging from 1 MPa to 4 MPa and all ranges and pressures there between (e.g., 1 MPa, 1.1 MPa, 1.2 MPa, 1.3 MPa, 1.4 MPa, 1.5 MPa, 1.6 MPa, 1.7 MPa, 1.8 MPa, 1.9 MPa, 2 MPa, 2.1 MPa, 2.2 MPa, 2.3 MPa, 2.4 MPa, 2.5 MPa, 2.6 MPa, 2.7 MPa, 2.8 MPa, 2.9 MPa, 3.0 MPa, 3.1 MPa, 3.2 MPa, 3.3 MPa, 3.4 MPa, 3.5 MPa, 3.6 MPa, 3.7 MPa, 3.8 MPa, 3.9 MPa, or 4 MPa). Preferably, the CO₂ pressure is about 2.5 MPa to 3.5 MPa. The upper limit on pressure can be determined by the type and size of reactor used. Although not shown, in some embodiments, CO₂CO, O₂, or H₂, and can be provided to reactor unit 202 via the same inlet. In certain embodiments, mixtures of CO₂, CO, O₂, and H₂ are used. By way of example, CO₂ can be used with CO, CO₂ can be used with H₂, CO, or CO and H₂, and CO can be used with O₂. Reactor 202 can be pressurized either through the addition of the gases and/or with an inert gas. The average pressure of reactor unit 202 can range from 2.0 to 4 MPa (e.g., 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9 MPa) after charging the CO₂. Reactor 202 can be heated to a temperature sufficient to promote the reaction of cesium carbonate with the carbon dioxide and carbon monoxide or H₂ to produce a product composition that includes cesium oxalate. The temperature range of the reactor 202 can be 200° C. to 400° C., 250° C. to 350° C., and all ranges and temperatures there between (e.g., 205° C., 210° C., 215° C., 220° C., 225° C., 230° C., 235° C., 240° C., 245° C., 250° C., 255° C., 260° C., 265° C., 270° C., 275° C., 280° C., 285° C., 290° C., 295° C., 300° C., 30520 C., 310° C., 315° C., 320° C., 325° C., 330° C., 335° C., 340° C., 345° C., 350° C., 355° C., 360° C., 365° C., 370° C., 375° C., 380° C., 385° C., 390° C., or 395° C.). Preferably, the reaction temperature is 290° C. to 335° C., or most preferably 300° C. to 325° C. The reactants can be heated for a time sufficient to react all or a substantially all of the cesium carbonate. By way of example, the reaction time range can be at least 1 hour, 1 to 5 hours, 1 hours to 4 hours, 1 hour to 3 hours, and all ranges and times there between (e.g., 1.25 hours, 1.5 hours, 1.75 hours, 2 hour, 2.25 hours, 2.5 hours, 2.75 hours, 3 hours, 3.25 hours, 3.5 hours, 3.75 hours, 4 hours, 4.25 hours, 4.5 hours, 4.75 hours, 5 hours). When CO is used, the reaction time can be about 2 hours. When H₂ is used, the cesium carbonate can be reacted with the carbon dioxide for 1 to 3 hours, (e.g., 1, 1.5, 2, 2.5, 3 hours), and then with H₂ for an additional 1 to 3 hours, (e.g., 1, 1.5, 2, 2.5, 3 hours). The metal oxalate (e.g., cesium oxalate) can be removed via solids outlet 210.

### 2. Alcohols

Alcohols may be purchased in various grades from commercial sources. Preferably the alcohol is devoid of, or includes a minimal amount, of water. Non-limiting examples of the alcohol that can be used in the process of the current invention to form a higher carbon number alcohol can include methanol, ethanol, n-propanol, isopropanol, n-butanol, isobutanol, sec-butanol, tert-butanol, 1-pentanol, 2-pentanol, 3-pentanol, 3-methyl-1-butanol, 2-methyl-b 1-butanol, 2,2-dimethyl -1-propanol, 3-methyl-2-butanol, 2-methyl-2-butanol, 1-hexanol, 2-hexanol, 3-hexanol, 1-heptanol, 2-heptanol, 3-heptanol, 4-heptanol, 1-octanol, 2-octanol, 3-octanol, 4-octanol, cyclohexanol, cyclopentanol, phenol, benzyl alcohol, ethylene glycol, propylene glycol, or butylene glycol or any combination thereof. Preferably, the alcohol is methanol.

### 3. Gases

CO₂ gas, CO gas, O₂ gas, and H₂ gas can be obtained from various sources. In one non-limiting instance, the CO₂ can be obtained from a waste or recycle gas stream (e.g., from a plant on the same site such as from ammonia synthesis, or a reverse water gas shift reaction) and/or after recovering the carbon dioxide from a gas stream. A benefit of recycling carbon dioxide as a starting material in the process of the invention is that it can reduce the amount of carbon dioxide emitted to the atmosphere (e.g., from a chemical production site). The CO can be obtained from various sources, including streams coming from other chemical processes, like partial oxidation of carbon-containing compounds, iron smelting, photochemical process, syngas production, reforming reactions, and/or various forms of combustion. O₂ can come from various sources, including streams from water-splitting reactions and/or cryogenic separation systems. The hydrogen may be from various sources, including streams coming from other chemical processes, like water splitting (e.g., photocatalysis, electrolysis, or the like), syngas production, ethane cracking, methanol synthesis, and/or conversion of methane to aromatics. In some embodiments, the gases are obtained from commercial gas suppliers. In some examples, the remainder of the reactant gas can include another gas or gases provided the gas or gases are inert, such as argon (Ar) and/or nitrogen (N₂), further provided that they do not negatively affect the reaction. Preferably, the reactant mixture is highly pure and substantially devoid of water. In some embodiments, the gases can be dried prior to use (e.g., pass through a drying media) or contain a minimal amount of water or no water at all. Water can be removed from the reactant gases with any suitable method known in the art (e.g., condensation, liquid/gas separation, etc.).

### 4. Cu-Based Catalysts

The catalyst can be any copper-based catalyst. The Cu-based catalyst can include a second transition metal (e.g., Columns 6-12 of the Periodic Table) and an optional promoter metal from Columns 1 and 2 of the Periodic Table. Non-limiting examples of transition metals include chromium, molybdenum, tungsten, manganese, iron, ruthenium, cobalt, rhodium, nickel, palladium, platinum, silver, gold, zinc, or a combination thereof. In one instance, the catalyst is a Cu-nickel (Ni) alloy. In some embodiments, the catalyst can be supported. Non-limiting examples of support material include silica, alumina, titania, magnesia, zirconia, carbon, or combinations thereof. Cu-based catalysts can be made using known synthetic methods or purchased from commercial sources. In some embodiments, a Cu-based metal precursor materials are co-precipitated over a support material. For example, a Cu precursor and a Ni precursor can be co-precipitated over a silica support using catalyst preparation methodology known in the art.

### EXAMPLES

The present invention will be described in greater detail by way of specific examples. The following examples are offered for illustrative purposes only, and are not intended to limit the invention in any manner. Those of skill in the art will readily recognize a variety of noncritical parameters, which can be changed or modified to yield essentially the same results.

### Example 1

### (Synthesis 35 wt % Cu Ni/SiO₂)

The 35 wt % Cu Ni/SiO₂ was prepared using precipitation methodology. Solution 1 was prepared by dissolving sodium carbonate (2.21 g) in water (150 mL). Solution 2 was prepared by mixing nickel nitrate hexahydrate (0.74 g, Ni(NO₃)₂.6H₂O), copper nitrate trihydrate (Cu(NO₃)₂.3H₂O, 6.65 g) and silica gel (3.1 g) in water (100 mL). Solution 2 agitated (420 rpm) for 1 hr. Solution 1 was titrated into Solution 2 until a blue precipitate appeared. The precipitate was filtered and washed with water several times. The solid precipitate was dried overnight at 120° C. and calcined at 450° C. for 4 hrs to obtain the final catalyst material.

### Example 2

### (Reduction of Cu-Ni Catalyst)

A known weight (1.000 g, 6.26 mM weight of Cu) of synthesized Cu-Ni catalyst (Example 2) was weighed into 100 mL Parr autoclave. The autoclave was pressurized to 3 MPa with H₂, and then heated to the reduction temperature of about 265° C. for 6 hours. After the completion of reduction, the reactor was cooled down to room temperature and depressurized to atmospheric pressure.

### Example 3

### (Production of Ethanol)

Methanol (25 mL) and cesium oxalate (1 g, 2.8 mM g, MilliporeSigman, USA) was added into to the reactor containing reduced Cu-catalyst of Example 2. The vessel was pressurized with 2 MPa CO₂ and 2 MPa H₂ at room temperature. Then the reactor was then heated to 220° C. with a continuous stirring for 15 hours. On completion of the reaction, the was depressurized to 25° C. and 0.1 MPa (atmospheric pressure). The reactor vessel was opened and a small portion of the sample product was characterized using ¹³C & ¹H NMR and by GC-MS. The product mixture included 30% ethanol in 30% and 10% ethylene glycol.

1. A process for producing an alcohol, the process comprising contacting a metal oxalate salt with a first alcohol in the presence of a copper (Cu)-based catalyst under conditions sufficient to produce a composition comprising a second alcohol having an increased carbon number relative to the first alcohol.

2. The process of claim 1, wherein the first alcohol is methanol and the second alcohol is ethanol.

3. The process of claim 1, wherein contacting is performed in a carbon dioxide (CO₂) and hydrogen (H₂) atmosphere.

4. The process of claim 1, wherein the Cu-based catalyst comprises nickel (Ni).

5. The process of claim 4, wherein the Cu-based catalyst is a supported Cu-Ni material.

6. The process of claim 1, wherein the metal oxalate salt to first alcohol molar ratio is 1 to 5.

7. The process of claim 1, wherein the reaction conditions comprise a temperature of 200° C. to 250° C.

8. The process of claim 1, wherein the metal oxalate salt comprises cesium (Cs).

9. The process of claim 8, wherein the metal oxalate salt is cesium oxalate (Cs₂C₂O₄).

10. The process of claim 9, wherein the cesium oxalate is obtained by contacting CO₂ and H₂ with cesium carbonate (Cs₂CO₃) under reaction conditions sufficient to form the Cs₂C₂O₄.

11. The process of claim 9, wherein the reaction conditions comprise a temperature of 250° C. to 400° C.

12. The process of claim 9, a CO₂ pressure of 1 MPa to 5 MPa and a H₂ pressure of 0.05 to 0.5 MPa.

13. The process of claim 8, wherein the mole ratio of CO₂ and H₂ to Cs₂CO₃ is 100:1 to 300:1.

14. The process of claim 1, further comprising the step of contacting a Cu-based catalyst precursor with gaseous hydrogen (H₂) under conditions sufficient to reduce the Cu-based catalyst precursor and form the Cu-based catalyst prior to contacting the Cu-based catalyst with the first alcohol.

15. The process of claim 14, wherein the conditions comprise a temperature of 250° C. to 280° C.

16. The process of claim 1, wherein the composition further comprises a diol.

17. The process of claim 16, wherein the diol is ethylene glycol.

18. The process of claim 16, wherein a mole ratio of second alcohol to diol is 10:1 to 20:1.

19. The process of claim 1, wherein the selectivity to the second alcohol is at least 10%.

20. The process of claim 1, wherein a molar ratio of the first alcohol to the Cu in the Cu-based catalyst is 200:1.
